(19) United States
(12) Patent Application Publication
(10) Pub. No.: US 2016/0081933 A1
(43) Pub. Date: Mar. 24, 2016

(54) PROCESS FOR STABILIZING THE DRUG RELEASE PROFILES OF POLYMER FILM COATED PHARMACEUTICAL COMPOSITIONS

(71) Applicants: Odette HENSEL, Weiterstadt (DE); Shraddha Sanjeev Joshi, Navi Mumbai (IN); Smitha SHETTY, Mumbai (IN); Takayuki MORITA, Singapore (SG); Tatsuya ISHII, Tsukuba-city, Ibaraki (JP); Hans-Ulrich PETEREIT, Darmstadt (DE)

(72) Inventors: Odette HENSEL, Weiterstadt (DE); Shraddha Sanjeev JOSHI, Navi Mumbai (IN); Smitha SHETTY, Mumbai (IN); Takayuki MORITA, Singapore (SG); Tatsuya ISHI, Tsukuba-city (JP); Hans-Ulrich PETEREIT, Darmstadt (DE)

(73) Assignee: EVONIK ROEHM GMBH, Darmstadt (DE)

(21) Appl. No.: 14/889,741

(22) PCT Filed: Aug. 19, 2013
(86) PCT No.: PCT/EP2013/067226

§ 371 Date: Nov. 6, 2015

(30) Foreign Application Priority Data
May 9, 2013 (IN) 2070/CHE/2013

(51) Int. Cl. A61K 9/16; A61K 31/138; A61K 31/522; C09D 133/12; A61K 9/28

(52) U.S. Cl. CPC A61K 9/167; C09D 133/12; A61K 9/1682; A61K 9/1635; A61K 9/2893; A61K 9/2846; A61K 31/522; A61K 31/138

(57) ABSTRACT

The invention relates to a process for stabilizing the drug release profile of a polymer film coated pharmaceutical composition, wherein the pharmaceutical composition comprises a core comprising an active pharmaceutical ingredient and a polymer film coating onto the core which was applied by a spray coating process, including a subsequent drying step, by applying microwave-irradiation, infrared-irradiation, UV-irradiation or ultra sonic wave irradiation to the polymer film coated pharmaceutical compositions.

The invention relates to a process for stabilizing the drug release profiles of polymer film coated pharmaceutical compositions, wherein the pharmaceutical composition comprises a core comprising an active pharmaceutical ingredient and a polymer film coating onto the core which was applied by a spray coating process including a subsequent drying process.

WO2006/010457 describes a method for producing pharmaceutical forms having a stable active ingredient release profile, the pharmaceutical forms having controlled release characteristics on account of a coating of vinyl (co)polymers. The method is characterized in that the coated pharmaceutical forms are conditioned at a temperature of 30 to 70° C. in a fluidized bed coating apparatus or a drum coating apparatus for at least 10 minutes until achievement of a stable active ingredient release profile, an atmospheric humidity of 5 to 30% being set during the conditioning. The stabilizing or conditioning results are comparable to the conventional stabilizing or conditioning (curing) of pharmaceutical forms in recirculating air drying cabinet for 24 hours at 40° C.

Wong T. W. describes in Current Drug Delivery, 2008, 5, p. 77-84, the Use of Microwave in Processing of Drug Delivery Systems. It is generally mentioned that the use of microwave opens a new route to control physicochemical properties of drug delivery profiles of pharmaceutical dosage forms. It is further mentioned that microwave may provide the intended release characteristics of drugs in dosage forms without the need for excessive heat, lengthy process and/or toxic reactants. However details for stabilizing the drug release profiles of polymer film coated pharmaceutical compositions are not disclosed.

### OBJECT OF THE INVENTION

It was an object of the invention to provide an alternative process for stabilizing the drug release profiles of a polymer film coated pharmaceutical composition, wherein the pharmaceutical composition comprises a core comprising an active pharmaceutical ingredient and a polymer film coating onto the core which was applied by a spray coating process including a subsequent drying step. The process should be simple and less time consuming than known processes.

The term “stabilizing of the drug release profile” is used synonymous to the term “conditioning of the drug release profile” or to the term “curing of the drug release profile”. The short terms are “stabilizing process”, “conditioning process” or “curing process” or simply “stabilizing”, “conditioning” or “curing”. “Uncured” means that no stabilizing, conditioning or curing process has been applied to the coated cores.

### ACHIEVEMENT OF THE INVENTION

The mechanisms of the film forming process after spray coating of pellet cores, granules, crystals or of tablet cores comprising, containing or consisting of an active ingredient with film forming polymeric coatings from dispersion containing polymeric latex particles is well known (s. for instance Chevlier Y. et al, Film formation with latex particles. Colliod Polym. Sci 1992; 270(8):806-821). The film forming process is usually distributed into two phases. The first phase is the so called coalescense phase in which the hydrophilic latex particle layer after it has been spayed onto pellet cores, granules, crystals or tablet cores comprising, containing or consisting of an active ingredient breaks up. This means that the single latex particles on the surface of these cores are fused to each other to a continuous film layer mainly by forces generated by the evaporating water. After this phase the mass of the water has been removed by evaporation and the polymer chains start an interdiffusion process. The water content during the interdiffusion process is almost constant. During this process the polymeric layer becomes more compacted and smoother on its surface. The interdiffusion process may take some time, for instance hours, days, weeks, months or even years before an end point has been reached. Since the structure of the polymeric layer changes constantly during this time, the drug release profiles also may vary and are usually not stable during storage. To achieve stable drug release profiles it is necessary to speed up the interdiffusion process by a stabilizing, conditioning or curing process.

The invention relates to process for stabilizing the drug release profiles of polymer film coated pharmaceutical compositions by interdiffusion, wherein the pharmaceutical compositions comprise a core comprising an active pharmaceutical ingredient and a polymer film coating onto the core which was applied by a spray coating process including a subsequent drying step. Interdiffusion and complete film formation is enhanced by applying microwave-irradiation, infrared-irradiation, UV-irradiation or ultrasonic waves to the polymer film coated pharmaceutical composition.

### Controlled Release Characteristics

Preferably the film coated pharmaceutical compositions have polymer film coatings with controlled release characteristics. For instance polymers like gelatine do not display controlled release characteristics. Preferably the polymer coatings comprise vinyl (co)polymers. Controlled release characteristics may be achieved when the film coatings are applied to the cores as films which are smooth, tight and having a sufficient thickness. The film coatings shall be totally or almost free from holes or pores that could interfere by diffusion effects with the controlled release characteristics of the polymer film coatings.

The polymer film coatings may further comprise excipients such as antioxidants, brighteners, binding agents, emulsifiers, flavouring agents, flow aids, fragrances, glidants, penetration-promoting agents, pigments, plasticizers, polymers or stabilizers. The amounts of the excipients must be in a range where the drug release characteristics are still essentially controlled by the properties of the polymer film coating. Thus excessive amounts of excipients which may cause inequalities or pores in the film structure should be avoided.

The term “controlled release characteristics” is well known to a person skilled in the art. The person skilled in the art understands under this term, for example, that the release profile for certain active ingredients can be tailored reproducibly to the active ingredient by means of the formulation of the pharmaceutical form, in particular by the choice of the external coating. Known “controlled” release characteristics are the pH controlled release of active ingredient or the diffusion controlled delayed, also designated as retarded, release of active ingredient. After the release of the pharmaceutical form for sale, the release profile must no longer significantly change even after relatively long storage, in order to guarantee the therapeutic action in vivo. Standardized methods for the determination of the release profile are adequately known to the person skilled in the art. USP35-NF30 <711> Dissolution and <724> Drug Release describe methods for the determination of the release profile and permissible tolerances.

The term “core” or “cores” shall include pellet cores, granules, crystals or tablet cores comprising, containing or consisting of an active ingredient, which may be a pharmaceutical active ingredient or a nutraceutical active ingredient or combinations thereof.

Coatings of vinyl (co)polymers which impart controlled release characteristics to pharmaceutical forms are adequately known. Mention may be made, in particular, of coatings of vinyl (co)polymers of the type comprising or consisting of the (meth)acrylate (co)polymers or comprising or coatings of the type consisting of the polyvinyl acetates including the derivatives of polyvinyl acetate. As a result of their pH-independent, slowly swelling characteristic in intestinal juice, coatings of (meth)acrylate copolymers having neutral radicals, (meth)acrylate copolymers having functional quaternary amino groups, and coatings of polyvinyl acetates cause a retarding, controlled release of active ingredient. As a result of the alkaline medium of the intestine, coatings of (meth)acrylate copolymers having anionic functional groups (carboxylic side groups) cause a pH-controlled, controlled release of the active ingredient. Coatings of (meth)acrylate copolymers having tertiary amino side groups serve for taste isolation and dissolve in the acidic medium of the stomach and effect a pH-controlled, rapid controlled release of the active ingredient.

The process is in particular suitable for pharmaceutical forms having coatings comprising vinyl (co)polymers, which have been applied from aqueous dispersions. The process is furthermore suitable, in particular, for pharmaceutical forms having coatings which cause a retarding or sustained release of the active ingredient.

### Stable Active Ingredient Release Profile

The disclosed process for stabilizing the drug release profiles of polymer film coated pharmaceutical compositions results in a stable active ingredient release profile during storage over a certain period of time for example 1 month under controlled climate conditions within or without packaging.

Within the meaning of USP35-NF30 <711> Dissolution and <724> Drug Release, a deviation of more than +/−10% of the declared amount of active ingredient for the respective pharmaceutical form or individual dose can be regarded as a significant, non tolerable change, which can lead in vivo to a modified therapeutic action. In this case, the pharmaceutical form could be described as unstable. The term “stable active ingredient release profile” is therefore defined as a person skilled in the art understands it taking the USP-NF into consideration.

A stable active ingredient release profile within the meaning of the invention is understood as meaning an active ingredient release profile which, compared to an active ingredient release profile of a reference preparation which has been conditioned in a recirculating air drying cabinet for 24 hours at 40° C. at an air humidity in the range of 30-70%, differs by not more than +/−10%. The % data relate here to the initial active ingredient content of the individual dose for the respective pharmaceutical form at the time directly after curing being representative for the time directly after curing at defined measurement times.

For example, for a polymer film coated pharmaceutical form “X” in tablet form, the amount of active ingredient “Y” can be declared for a tablet. At the times “T1” and “T2” and “T3”, in each case specified aliquot amounts “Z1”, “Z2” and “Z3” of the amount of active ingredient “Y” must then be released under defined conditions. Permissible deviations at the times indicated, starting from the respective theoretical value of the indicated aliquot amounts “Z1”, “Z2” and “Z3”, are not more than +/−10% of the amount of active ingredient “Y”. If, for example, the value “Z2” is 50%, values in the range from 40 to 60% are to be regarded as stable.

The active ingredient release curve of a pharmaceutical form having a retarding polymer coating type can be recorded, for example, for 4 to 12 hours at a constant pH. The active ingredient release curve of a pharmaceutical form having an anionic polymer coating type is as a rule only recorded for 2 hours at pH 1.2, for the gastric juice resistance test, and subsequently for a number of hours at a constant higher pH, for the active ingredient release test. The release curve of a pharmaceutical form having a cationic polymer coating type containing tertiary amino groups can be recorded, for example, at a constant pH over a short time of 10 to 60 at least, since these coating types dissolve comparatively rapidly.

### Preparation of Polymer Film Coated Pharmaceutical Forms

In a manner known per se, active ingredient-containing cores or pellet cores form the basis for the polymer coatings, preferably for coatings comprising vinyl (co)polymers. Pelletizing can be carried out on active ingredient-free spheres (nonpareils) or core-free pellets, pellet cores, can be produced.

First, a rounded, active ingredient-containing substrate with or without a neutral carrier is produced. By means of a fluidized bed process, liquid can be applied to placebo pellets or other suitable carrier materials, the solvent or suspending agent being evaporated. According to the preparation process, a drying step, for instance for about 5 minutes, can be added. Alternatively commercially available drug containing cores can be used to be coated.

The still uncoated, rounded layer is designated, for example, as the core or as a pellet. Suitable sizes of a core may be between 50 to 1800 μm or 50-1000 μm average diameter. The active ingredient is as a rule brought into an organic solvent or into water and mixed. In order to provide a satisfactory ability of the mixture to be sprayed, it is usually necessary to formulate a mixture with relatively low viscosity. The addition of a detergent, e.g. Tween®, in concentrations of 0.1 to 20, preferably 0.5 to 10% by weight, may be advantageous for the reduction of the surface tension. In addition to the active ingredient, the core may comprise further pharmaceutical excipients such as binders, for instance cellulose and its derivatives or polyvinylpyrrolidon (PVP), moisture retention agents, disintegration promoters, lubricants, disintegrants, (meth)acrylates, starch and its derivatives, sugar solubilizers or others.

Appropriate application processes are known from textbooks, for example, from: Bauer, Lehmann, Osterwald, Rothgang “Uberzogene Arzneiformen” [Coated Pharmaceutical Forms] Wissenschaftliche Verlagsgesellschaft mbH Stuttgart, Chap. 7, pp. 165-196. Details are furthermore known to the person skilled in the art from textbooks. See, for example: Voigt, R. (1984): Lehrbuch der pharmazeutischen Technologie [Textbook of Pharmaceutical Technology]; Verlag Chemie Weinheim—Beerfield Beach/Florida—Basle. Sucker, H., Fuchs, P., Speiser, P.: Pharmazeutische Technologie [Pharmaceutical Technology], George Thieme Verlag Stuttgart (1991), in particular chapters 15 and 16, pp. 626-642. Gennaro, A., R. (Editor), Remington's Pharmaceutical Sciences, Mack Publishing Co., Easton Pa. 30 (1985), Chapter 88, pp. 1567-1573. List, P. H. (1982): Arzneiformenlehre [Pharmaceutical Form Theory], Wissenschaftliche Verlagsgesellschaft mbH, Stuttgart.

Cores or pellet cores can be rounded by processes such as wet granulation with subsequent spheronizing, rotoragglomeration, precipitation, spray drying, melt extrusion or spray processes, in particular ultrasonic vortex spray processes, to give still uncoated cores or pellets of defined size, e.g. 50 to 1800 μm or 50 to 1000 μm. This has the advantage that the entire core volume is available for active ingredient loading. The active ingredient loading can thereby again be increased in relation to the embodiment having an inert core.

After preparation of the active ingredient-containing cores or pellet cores, these are provided in spray processes with an outer polymer film coating, preferably a coating of vinyl (co)polymers, such that coated pellets are obtained. The pellets are prepared by means of spray application from organic solution, or preferably from aqueous dispersions. For implementation, it is crucial here that uniform, almost or totally pore-free coatings should result. This ensures that the drug release profiles of polymer film coated pharmaceutical compositions are due to the functionally of their coatings rather than being influenced by undesired artificial effects.

The equipment suitable for spray coating, the fluidized bed coating apparatus (fluidized bed coater) or the drum coating apparatus (drum coater) are known to the person skilled in the art from galenics.

In the fluidized bed coating apparatus, active ingredient-containing cores or active ingredient-containing pellets can be provided, for example, with a coating of vinyl (co)polymers. For this purpose, the active ingredient-containing cores or active ingredient-containing pellets are fluidized in a stream of air to give a permanent to and fro movement, while the (meth)acrylate copolymer is simultaneously sprayed in the form of a finely nebulized dispersion. The polymer dispersion precipitates on the active ingredient-containing cores or active ingredient-containing pellets and forms a film there. The water contained evaporates in the stream of air, in which, as a rule, an inlet air temperature in the range from 20° C. to 70° C., preferably 30 to 60° C. is set.

In a drum coating apparatus (drum coater), the movement of the active ingredient-containing cores or active ingredient-containing pellets takes place by means of the movement of the drum.

As a rule, the coated pellets are additionally subsequently dried for a few minutes, for instance for about 3 to 8, preferably 4 to 6 or for 5 minutes, after the spray application before the stabilizing, curing or conditioning process is begun.

### Residual Water Content of the Polymer Film Coated Pharmaceutical Compositions

The spray coating process is including a subsequent drying step. At this stage the water content of the uncured material or pellets is already lowered to a rather low residual water content. Usually this residual water is further not or only very little lowered by the following stabilizing or curing procedure as described in here. Thus the stabilizing or curing procedure should be not regarded as a drying step but rather as a step in which interdiffusion processes in the polymer structure occur.

The residual water content of the polymer film coated pharmaceutical compositions which drug release profiles are to be stabilized may be in the range of less than 10, in the range of 0.3 to 10, preferably from 1 to 5% by weight. The difference of the water content of uncoated pellet or core and the coated pellet or core should be no more or less than plus/minus 5, 3 or preferably 1% before the conditioning, stabilizing or curing process.

The residual water content may be analyzed according to USP 35-NF30 <921> Water Determination or by “Karl Fischer titration”, using a 831 KF-coulometer by Deutsche METROHM GmbH & Co. KG (Filderstadt, Germany) or other suitable equipment. The term and the method of the USP 35-NF30 <921> Water Determination and the term and the method of the “Karl Fischer titration” are well known to a person skilled in the art.

The residual water content may be also analyzed by the loss on drying (LOD) method. The loss on drying (LOD) of the polymer film coated pharmaceutical compositions which drug release profiles are to be stabilized may be in the range of less than 5, in the range of 0.3 to 5, preferably from 1 to 4% by weight. The difference of the loss on drying of uncoated pellet or core and the coated pellet or core should be no more or less than plus/minus 5, 3 or preferably 1% before the conditioning, stabilizing or curing process.

For the determination of the loss on drying (LOD) a defined amount of crushed tablets or particles (uncoated or coated tablets or uncoated or coated pellets), e.g. 3 g, are to be heated up to a defined temperature, e.g. 110° C., and to be dried until a mass constancy will be achieved, using the Moisture Analyzer HG 63 by Mettler-Toledo GmbH (Gieβen, Germany) or other suitable equipments. The loss on drying is the weight difference after and before drying in % of the sample weight before heating. The term and the method of the USP 35-NF30 <731> Loss On Drying are well known to a person skilled in the art.

### The Stabilizing Process

The process for stabilizing the drug release profiles of polymer film coated pharmaceutical compositions as disclosed may be also called a stabilizing process, a conditioning process or a curing process.

An often applied stabilizing process is air oven curing for 24 hours at 40° C. Therefore air oven curing for 24 hours at 40° C. is taken as a reference method in the examples. The stabilizing results achieved according to this invention, the resulting active ingredient release profiles, are in the same range as the air oven curing results but much less time is needed to perform.

### Microwave-Irradiation

The invention discloses a process, wherein microwave-irradiation with frequencies within the range of 300 MHz to less than 300 GHz (wavelength 1 mm-1 m) may be applied to the polymer film coated pharmaceutical compositions in order to stabilize their drug release profiles.

The principle of microwave is well described by Wong T. W. describes in Current Drug Delivery, 2008, 5, p. 77-84, the Use of Microwave in Processing of Drug Delivery Systems, which is incorporated hereby by reference. According to Wong microwave is generated by a magnetron, which converts electrical energy into an alternating electrical field. The magnetron usually consists of four components: an anode block, a cathode filament, a pair of permanent magnets and an antenna. The production of microwave begins when an electron is emitted by the cathode filament and is accelerated towards the anode block. The electron is making spiral movements under the influences of applied electric and magnetic fields. The electric field alternates the charge and the magnetic fields at high frequencies. The current is sampled by an antenna and released as microwave through a waveguide.

The invention discloses a process, wherein microwave-irradiation preferably in the range of 2.4 to 2.5 GHz, preferably around or at 2.45 GHz is applied.

The invention discloses a process, wherein microwave-irradiation in the range of 2.4 to 2.5 GHz, preferably around or at 2.45 GHz, is applied to batches of polymer film coated pharmaceutical compositions, preferably by means of a microwave apparatus with a size and performance rate suitable for domestic applications, at 300 to 1500 Watt for 1 to 10 minutes as a rough rule.

A microwave equipment or apparatus with a size and performance rate suitable for domestic applications may be suitable for preparing heating meals and may for instance have a housing with an irradiation chamber of a size of about 20-60 cm in broadness, length and height wherein the product may be irradiated at a distance of around 15 to 55, preferably 15 to 25 cm with for instance 50 to 1500, preferably 300 to 1000 Watt for a few seconds or up to several minutes.

Suitable irradiation conditions for the inventive process may be for example 100, 200, 300, 400, 500, 600, 700, 800, 900 or 1000 Watt for 30 seconds or for 1, 2, 3, 4, 5, 6, 7, 8, 9 or 10 minutes and each and every possible combination, values in between or ranges that can be derived from the values given here. A suitable working range may be 300-1200, 400-1000 Watt. A suitable application time may be for 1 to 8, preferably 1.5 to 6 minutes. Good and acceptable results including appropriate storage stability may be achieved at 400-630 Watt for 1-4 minutes.

Microwave curing may be shortened with higher energy input.

The product temperature in the microwave equipment may vary from 30-160, preferably from 40-120, 50 to 100 or from 60 to 80° C.

A person skilled in the art of pharmacy or galenics is able to select or to find out suitable stabilizing conditions depending on the certain microwave equipment or apparatus and the pharmaceutical compositions to be stabilized.

Suitable commercially available microwave equipment is for instance SHARP R-939-A; frequency 2450 MHz; preferably using 900 Watt as a product position: central; the distance between microwave source and product is approximately 20 cm; the product temperature may vary from 30-160° C.

### Infrared-Irradiation

The invention discloses a process, wherein infrared-irradiation with frequencies within the range of 300 GHz to 400 THz (wavelength 780 nm-1 mm) may be applied to the polymer film coated pharmaceutical compositions in order to stabilize their drug release profiles.

A person skilled in the art of pharmacy or galenics is able to select or to find out suitable stabilizing conditions depending on the certain equipment or apparatus and the pharmaceutical compositions to be stabilized.

Suitable equipments for applying ultra infrared-irradiation are IR lamp, IR dryer, IR spectrometer, IR radiator or IR heater.

A suitable commercially available model is for instance InfraCare Phillips HP3621; preferable using 200 Watt; 750 Watt/m²; product position: central; the distance between lamp and product is preferable in a range of 8-15 cm; the product temperature may vary from 35-70° C. Suitable curing times may be 2-40, 3-20 or 3-14 minutes.

Curing conditions may be optimized according to physical properties of the sample particularly according to the type of polymer and the formulation of the coating composition.

### UV-Irradiation

The invention discloses a process, wherein UV-irradiation with wave lengths within the range from 789 THz to 300 PHz, (wavelength 1 nm-200 nm) may be applied to the polymer film coated pharmaceutical compositions in order to stabilize their drug release profiles.

A person skilled in the art of pharmacy or galenics is able to select or to find out suitable stabilizing conditions depending on the certain ultrasonic wave equipment or apparatus and the pharmaceutical compositions to be stabilized.

Suitable equipments for applying ultra UV-irradiation are UV lamp, UV radiator, UV heater, UV dryer or UV spectrometer.

A suitable commercially available model is for instance Safety cabinet HERA Safe 2020 (Thermo Scientific); the distance between lamp and product: approximately 5-8 cm; the product temperature: room temperature may vary from 22-24° C. Suitable curing times may be 2-150, 3-130, 18-150 or 20-130 minutes.

Curing conditions may be optimized according to physical properties of the sample particularly according to the type of polymer and the formulation of the coating composition.

### Ultrasonic Waves/Ultra Sonic Wave Irradiation

The invention discloses a process, wherein ultrasonic waves (ultra sonic wave irradiation) with frequencies within the range 16 kHz to 1 GHz may be applied to the polymer film coated pharmaceutical compositions in order to stabilize their drug release profiles. The wavelength of the ultrasonic waves respectively the ultra sonic wave irradiation depends on the medium and the density of the medium in which the curing is carried out (e.g. in air or water).

The invention discloses a process, wherein ultrasonic-irradiation preferably in the range of 20 kHz to 2 MHz, preferably between 20 kHz and 400 kHz or especially in the range of 35-200 kHz is applied.

Suitable ultrasonic wave conditions for the inventive process may be for example at 30, 40, 50, 60, 70, 80 or 90° C. or 30-90 or 30-50° C. for 30 seconds, 1, 2, 3, 4, 5, 6, 7, 8, 9 or 10 minutes or for 0.5-150 or 1-150 minutes and each and every possible combination of these values, including values in between or ranges that can be derived from the values given here.

Curing conditions may be optimized according to physical properties of the sample particularly according to the type of polymer and the formulation of the coating composition. Additionally curing periods will be influenced by temperature interaction by the sample.

A person skilled in the art of pharmacy or galenics is able to select or to find out suitable stabilizing conditions depending on the certain ultrasonic wave equipment or apparatus and the pharmaceutical compositions to be stabilized.

Suitable equipment for applying ultra sonic waves or ultra sonic wave irradiation is an ultrasound bath with water as medium: A suitable commercially available model is for instance Bandelin SONOREX® SUPER RK 514 BH; frequency 35 kHz; 450 Watt.

### Application Times

The process disclosed herein is characterized in that the microwave-irradiation, the infrared-irradiation, the UV-irradiation or the ultrasonic wave irradiation is applied to the polymer film coated pharmaceutical composition for not more than 2, not more than 5, not more than 10, not more than 20, not more than 30, not more than 40, not more than 50 or not more than 60 minutes.

### Further Embodiments

The stabilized or conditioned coated cores obtained can preferably be further processed by means of pharmaceutically customary excipients and in a manner known per se to give a multiparticulate pharmaceutical form, in particular to give pellet-containing tablets, minitablets, capsules, sachets or inspissated juices.

The process can be carried out in a separate microwave-irradiation device, infrared-irradiation device, UV-irradiation device or ultrasonic wave device after the coating and drying procedure in a fluidized bed coating apparatus or in a drum coating apparatus.

The process can be further carried out in a fluidized bed coating apparatus or in a drum coating apparatus, which is additionally equipped with microwave-irradiation devices, infrared-irradiation device, UV-irradiation devices or ultrasonic wave devices, where the cores (pellets, granules, crystals or tablet cores comprising, containing or consisting of an active ingredient) are coated first, dried and then stabilized or conditioned in the same apparatus.

### Coatings Comprising Neutral Vinyl (Co)Polymers

### Neutral (Meth)Acrylate Copolymers, EUDRAGIT® NE or EUDRAGIT® NM Type

The process according to the invention is suitable for the conditioning of pharmaceutical forms whose coatings are comprising or consisting essentially of or consisting of (meth)acrylate (co)polymers which are polymerized to more than 95% by weight to 100% from monomers containing neutral radicals. Monomers containing neutral radicals can in particular be C1- to C4-alkyl esters of acrylic or methacrylic acid are in particular methyl methacrylate, ethyl methacrylate, butyl methacrylate, methyl acrylate, ethyl acrylate and butyl acrylate.

Mention may be made, for example, of neutral (meth)acrylate copolymers comprising or consisting of 20 to 40% by weight of ethyl acrylate and 60 to 80% by weight of methyl methacrylate (EUDRAGIT® NE type). Optionally, the largely neutral (meth)acrylate copolymers mentioned can contain small proportions, e.g. 0 to less than 5, preferably 0 to 2% by weight, of (meth)acrylate monomers having an anionic group in the alkyl radical, for example acrylic acid, but preferably methacrylic acid. The mainly or completely neutral copolymer preferably has the property of swelling above pH 5.0 in water or in the intestinal juice medium and releasing active ingredient.

EUDRAGIT® NE is a copolymer of 30% by weight of ethyl acrylate and 70% by weight of methyl methacrylate. The polymer can be employed, for example, in the form of a 30% strength aqueous dispersion, EUDRAGIT® NE 30D.

### Methacrylate Copolymers Containing Anionic Radicals Respectively Carboxylic Side Groups.

The process is suitable for pharmaceutical forms comprising or consisting essentially of or consisting of coatings of (meth)acrylate copolymers which consist to 25 to 95% by weight of free radical-polymerized C1- to C4-alkyl esters of acrylic or methacrylic acid and to 30 to 75% by weight of (meth)acrylate monomers containing an anionic group in the alkyl radical respectively carboxylic side groups.

C1- to C4-alkyl esters of acrylic or methacrylic acid are, in particular methyl methacrylate, ethyl methacrylate, butyl methacrylate, methyl acrylate, ethyl acrylate and butyl acrylate. A (meth)acrylate monomer containing an anionic group in the alkyl radical respectively a carboxylic side group can be, for example, acrylic acid, but preferably methacrylic acid.

As a rule, the proportions mentioned add up to 100% by weight. However, without leading to an adverse effect on or change in the essential properties, small amounts in the range from 0 to 10, e.g. 1 to 5% by weight, of further vinylically copolymerizable monomers, such as, for example, hydroxyethyl methacrylate or hydroxyethyl acrylate, may be additionally be present.

### EUDRAGIT® L100, L100-55, S100 and FS Coating Types.

The process is suitable for pharmaceutical forms comprising or consisting essentially of or consisting of coatings of (meth)acrylate copolymers consisting of 40 to 60% by weight of methacrylic acid and 60 to 40% by weight of methyl methacrylate or 60 to 40% by weight of ethyl acrylate (EUDRAGIT® L100 or EUDRAGIT® L100-55 types).

EUDRAGIT® L is a copolymer of 50% by weight of methyl methacrylate and 50% by weight of methacrylic acid. EUDRAGIT® L 30D is a dispersion comprising 30% by weight of EUDRAGIT® L.

EUDRAGIT® L100-55 is a copolymer of 50% by weight of ethyl acrylate and 50% by weight of methacrylic acid. EUDRAGIT® L 30-55 is a dispersion comprising 30% by weight of EUDRAGIT® L 100-55.

The process is suitable for pharmaceutical forms comprising coatings of (meth)acrylate copolymers consisting of 20 to 40% by weight of methacrylic acid and 80 to 60% by weight of methyl methacrylate (EUDRAGIT® S100 type).

The process is suitable for pharmaceutical forms comprising coatings of (meth)acrylate copolymers consisting of 10 to 30% by weight of methyl methacrylate, 50 to 70% by weight of methyl acrylate and 5 to 15% by weight of methacrylic acid (EUDRAGIT® FS type). EUDRAGIT® FS is a copolymer of 25% by weight of methyl methacrylate, 65% by weight of methyl acrylate and 10% by weight of methacrylic acid. EUDRAGIT® FS 30D is a dispersion comprising 30% by weight of EUDRAGIT® FS.

### EUDRAGIT® Coating Types Having a Medium Content of Methacrylic Acid.

The process is suitable for pharmaceutical forms comprising coatings of (meth)acrylate copolymers consisting of anionic (meth)acrylate copolymers consisting of 20 to 34% by weight of methacrylic acid and/or acrylic acid, 20 to 69% by weight of methyl acrylate and 0 to 40% by weight of ethyl acrylate and optionally 0 to 10% by weight of further vinylically copolymerizable monomers, with the proviso that the glass transition temperature of the copolymer according to ISO 11357-2, item 3.3.3, is at most 60° C.

Such a copolymer may be in particular composed of free radical-polymerized units of 20 to 34, preferably 25 to 33, particularly preferably 28 to 32% by weight of methacrylic acid or acrylic acid; methacrylic acid is preferred, 20 to 69, preferably 35 to 65, particularly preferably, 35 to 55% by weight of methyl acrylate and optionally 0 to 40, preferably 5 to 35, particularly preferably 35 to 35% by weight of ethyl acrylate, with the proviso that the glass transition temperature of the copolymer (without plasticizer addition) according to ISO 11357-2, item 3.3.3, is at most 60, preferably 40 to 60, particularly preferably 45 to 55° C.

The (meth)acrylate copolymer may comprise, consist essentially or consist of the monomers methacrylic acid, methyl acrylate and ethyl acrylate in the quantitative proportions indicated above. As a rule, the proportions mentioned may add up to 100% by weight. However, without leading to an adverse effect on or change in the essential properties, small amounts in the range from 0 to 10, e.g. 1 to 5% by weight of further vinylically such as, for example, methyl methacrylate, butyl methacrylate, butyl acrylate or hydroxyethyl methacrylate may be additionally be present.

### Coatings Comprising (Meth)Acrylate Copolymer Containing Functional Cationic Radicals

### (Meth)Acrylate Copolymers Containing Tertiary Amino Groups, EUDRAGIT® E100 and EPO Type.

The process is suitable for pharmaceutical forms comprising or consisting essentially of or consisting of coatings comprising (meth)acrylate copolymers polymerized from 30 to 80% by weight of free radical-polymerized C1- to C4-alkyl esters of acrylic or of methacrylic acid and 70 to 20% by weight of (meth)acrylate monomers containing a tertiary amino group in the alkyl radical.

Suitable monomers containing functional tertiary amino groups are listed in U.S. Pat. No. 4,705,695, column 3, line 64 to column 4, line 13. In particular, mention may be made of dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethyl)propyl methacrylate, (3-diethylamino-2,2-dimethyl)propyl acrylate and diethyl amino-2,2-dimethyl)propyl methacrylate, diethylaminoethyl acrylate or diethylaminoethyl methacrylate. Dimethylaminoethyl methacrylate is particularly preferred.

The content of monomers containing tertiary amino groups in the copolymer may be between 20 and 70% by weight, preferably between 40 and 60% by weight. The proportions of the C1- to C4-alkyl esters of acrylic or methacrylic acid may be 80-30, preferably between 60 and 40% by weight. Suitable C1- to C4-alkyl esters of acrylic or methacrylic acid may be methyl methacrylate, ethyl methacrylate, butyl methacrylate, methyl acrylate, ethyl acrylate and/or butyl acrylate.

A customary (meth)acrylate copolymer containing tertiary amino groups may be synthesized, for example from 20-30% by weight of methyl methacrylate, 20-30% by weight of butyl methacrylate and 60-40% by weight of dimethylaminoethyl methacrylate.

A commercially customary (meth)acrylate copolymer containing tertiary amino groups may be synthesized, for example, from 25% by weight of methyl methacrylate, 25% by weight of butyl methacrylate and 50% by weight of dimethylaminoethyl methacrylate (EUDRAGIT® E100).

A further commercially customary (meth)acrylate copolymer containing tertiary amino groups is, for example, EUDRAGIT® E PO: Copolymer of methyl methacrylate, butyl methacrylate, and dimethylaminoethyl methacrylate in the ratio of 25:25:50 having a mean particle size of 15 μm.

### (Meth)Acrylate Copolymers Containing Quaternary Ammonium Groups, EUDRAGIT® RS or RL Type

The process is in particular suitable for pharmaceutical forms comprising or consisting essentially of or consisting of coatings of (meth)acrylate copolymers with quaternary ammonium groups, in particular of copolymers which are synthesized from free radical-polymerized units of 50-70% by weight of methyl methacrylate, 20-40% by weight of ethyl acrylate and 12-2% by weight of 2-trimethylammonium ethyl methacrylate chloride (EUDRAGIT® RS or RL type).

The process is in particular suitable for pharmaceutical forms comprising coatings of (meth)acrylate copolymers with quaternary ammonium groups, which were applied from aqueous dispersions.

The process is in particular suitable for pharmaceutical forms comprising coatings comprising (meth)acrylate copolymers which are synthesized from polymerized units of 65% by weight of methyl methacrylate, 30% by weight of ethyl acrylate and 5% by weight of 2-trimethylammonium ethyl methacrylate chloride (EUDRAGIT® RS).

The process is in particular suitable for pharmaceutical forms comprising coatings of (meth)acrylate copolymers which from free radical-polymerized units of 60% by weight of methyl methacrylate, 30% by weight of ethyl acrylate and 10% by weight of 2-trimethylammonium ethyl methacrylate chloride (EUDRAGIT® RL). The process is in particular suitable for pharmaceutical forms having coatings comprising mixtures of EUDRAGIT® RS and EUDRAGIT® RL.

### Vinyl Copolymers

The process is in particular suitable for pharmaceutical forms comprising or consisting essentially of or consisting of a (co)polymer coating which is polyvinyl acetate or a polyvinyl acetate.

The term “a polyvinyl acetate” additionally includes derivatives of polyvinyl acetate. The polyvinyl acetate can be present as a dispersion (e.g. of the type Kollicoat® SR 30 D, manufacturer BASF, polyvinyl acetate dispersion, stabilized with povidone and sodium laurylsulfate).

### EXAMPLES

### Curing of Films and Coated Medications with Microwave, Ultrasonic, IR and UV

### I. Curing Technology

### IR Lamp:

InfraCare Philips HP3621 by Philips Consumer Lifestyle B.V. (Drachten, Netherlands); 200 Watt; 750 Watt/m²; distance between lamp and product: 8-15 cm; product temperature: 35-70° C.; curing: open on glass petri dishes

### UV Lamp:

Safety cabinet HERA Safe 2020 (Thermo Scientific) by Thermo Electron LED GmbH (Langenselbold, Germany); distance between lamp and product: approximately 5-8 cm; product temperature: room temp. 22-24° C.; curing: closed in PMMA foil

### Microwave:

SHARP R-939-A by SHARP Electronics (Europe) GmbH (Hamburg, Germany); 900 Watt; product position: central; distance between microwave source and product: approximately 20 cm; product temperature: 30 −160° C.; curing: open on dishes suitable for microwave

Ultrasound Bath with Water (Aqua Pur) as Media:

Bandelin SONOREX SUPER RK 514 BH by BANDELIN electronic GmbH & Co. KG (Berlin, Germany); 450 Watt; curing: the product was sealed in PE-plastic bags and placed in the middle of the water bath completely under water; 40-80° C. water temperature.

### Air Oven:

EHRET TK/L 4250 by EHRET GmbH & Co. KG (Emmendingen, Germany); 3000 Watt; product position: central; curing: open on trays

The air oven curing for 24 hours at 40° C. is an often applied stabilizing process. Therefore air oven curing for 24 hours at 40° C. is taken as a reference method here in the examples.

### II. Example 1 (According to the Invention)

### Diprophylline Pellets Coated with EUDRAGIT® RS 30 D

1,200 g Dipropylline pellets (average diameter 800-1,000 μm; 50% drug content) by NBS Biologicals Ltd. (Huntingdon, Cambridgeshire UK) were coated with EUDRAGIT® RS 30D by Evonik Industries AG (Darmstadt, Germany), using the formulation of table 1.

TABLE 1
Coating formulation

| | [g] | [%] on polymer |
|---|---|---|
| EUDRAGIT ® RS 30D | 600.0 | |
| Triethyl citrate (TEC) | 36.0 | 20 |
| Glycerol monostearate (GMS) | 9.0 | 5 |
| Polysorbate 80 (33% aqu.) | 10.8 | 2 |
| Water (Aqua purificata) | 487.2 | |
| Total | 1,143.0 | |
| Solid content [%] | | 20 |
| Polymer applied [%] | | 15 |

50% water (Aqua purificata) was heated up to 80° C. Polysorbate 80 (Tween® 80) by Merck KGaA (Darmstadt, Germany), triethyl citrate by Merck KGaA (Darmstadt, Germany) and glycerol monostearate (Imwitor 900 K) by Sasol Germany GmbH (Witten, Germany) were homogenized in the heated water (aqua purificata) for 15 minutes, using a high shear force (e.g. Ultra Turrax). After 15 minutes the remaining amount of water was added to the suspension and then cooled down to room temperature while stirring with a conventional propeller stirrer. Afterwards the suspension was added into the polymer dispersion under permanently stirring. Finalized the suspension was passed through a 240 μm sieve to control that the suspension was free of agglomerates. During the coating process the suspension was gently stirred.

The coating parameters are listed in table 2.

TABLE 2
Equipment set up: Glatt GPCG 1.1 fluid bed coater, top spray mode by
Glatt Process Technology GmbH (Binzen, Germany)

| | | |
|---|---|---|
| Batch size core material | (g) | 1,200.0 |
| Nozzle bore | (mm) | 1.0 |
| Distance bed/spray gun | (cm) | ~10 |
| Internal tube diameter | (mm) | 2.0 |

Process parameter set up:

| | | |
|---|---|---|
| Atomizing air pressure | (bar) | 2.0 |
| Filter cleaning interval | (s) | 60 |
| Filter cleaning time | (s) | 5 |
| Drying air volume | (m³/h) | 60-80 |
| Drying air capacity | (m³/min/kg) | 1.2-1.6 |

Process data:

| | | |
|---|---|---|
| Inlet air temperature | (° C.) | 45-55 |
| Exhaust air temperature | (° C.) | 25-29 |
| Product temperature | (° C.) | 26-29 |
| Spray rate | (g/min/kg) | 7-11 |
| Spraying time | (min) | ~70 |
| Drying | (min) | 5 |

Table 3 shows the drug release profiles of cured coated pellets; using USP 35-NF30 <724> Drug Release and <711> Dissolution described methods for the determination of the release profile and permissible tolerances. The investigations of the drug release were carried out in an apparatus 2, e.g. Erweka DT 708/1000B by ERWEKA GmbH (Heusenstamm, Germany) in 0.1 N hydrochloric acid, pH 1.2, for the first 120 minutes, followed by phosphate buffer pH 6.8 at 100 rpm for the rest of the time. The amount of drug released was measured using an UV spectroscope with a wavelength of 273 nm in 914 ml total volume.

TABLE 3
Influence curing conditions on drug release [%]; initial values

| | Air oven | | | | | | | | |
|---|---|---|---|---|---|---|---|---|---|
| | curing | Microwave curing | | | Ultrasonic | | | | |
| Time | 24 h at | 450 W | 450 W | 900 W | 40° C. | 40° C. | 60° C. | 60° C. | 80° C. |
| [min] | 40° C. | 1 min | 3 min | 1 min | 30 min | 45 min | 3 min | 5 min | 1 min |
| 0 | 0.00 | 0.01 | 0.00 | 0.00 | 0.00 | 0.01 | 0.00 | 0.02 | 0.00 |
| 120 | 0.95 | 1.25 | 1.23 | 1.00 | 1.84 | 1.19 | 1.04 | 1.22 | 1.51 |
| 180 | 6.57 | 6.10 | 6.79 | 7.05 | 10.15 | 8.16 | 7.19 | 8.92 | 11.23 |
| 240 | 46.01 | 40.24 | 42.26 | 47.87 | 50.25 | 50.32 | 43.70 | 49.56 | 51.62 |
| 300 | 82.41 | 81.29 | 79.87 | 83.63 | 84.64 | 83.70 | 80.65 | 84.61 | 85.43 |
| 360 | 92.66 | 94.08 | 92.14 | 94.01 | 94.58 | 94.08 | 93.88 | 96.64 | 96.91 |
| 420 | 96.13 | 98.10 | 96.07 | 97.57 | 98.21 | 97.83 | 99.06 | 100.91 | 98.19 |
| 480 | 97.48 | 99.60 | 97.74 | 98.69 | 99.46 | 99.21 | 101.49 | 101.99 | 100.81 |

### Results:

Microwave curing with 450 Watt for 1 and 3 minutes, 900 Watt for 1 minute or Ultrasonic curing at 40° C. (temperature of the surrounding water) for 30 and 45 minutes, at 60° C. (temperature of the surrounding water) for 3 and 5 minutes as well as at 80° C. (temperature of the surrounding water) for 1 minute result in a very similar active ingredient release (diprophylline) as the standard method air oven curing 24 h/40° C. The deviation is less than ±10% of the drug content of the reference and therewith stabilized active ingredient release profiles were achieved with microwave irradiation or ultrasonic wave irradiation.

The loss on drying (LOD) as well as the residual water content (according to USP 35-NF30 <921> Water Determination) in table 4 confirm the curing effect and show that it is definitive a curing and not a drying process. The determination of the LOD was carried out with the Moisture Analyzer HG 63 by Mettler-Toledo GmbH (Gieβen, Germany). 3 g pellets were heated up to 110° C. and dried until mass constancy was achieved. The water content was analyzed via Karl Fischer (KF) titration, using the 831 KF-coulometer by Deutsche METROHM GmbH & Co. KG (Filderstadt, Germany).

A person skilled in the art of pharmacy or galenics knows, that the determination of the loss on drying (LOD) is quite easier and faster compared to the determination of the water content by Karl Fischer titration and that differences between both methods are caused by the principle of measurement (titration chemical method, loss on drying physical method), but do not effect their meaningful relationship.

At a loss on drying (LOD) below 1% the correlation to the residual water content (KF) is between 2-3 fold. At a loss on drying above 1% the correlation between loss on drying (LOD) and residual water content (KF) tends to be narrower.

TABLE 4
LOD and water content [%]

| | | Residual Water |
|---|---|---|
| | LOD [%] | content KF [%] |
| uncured | 0.60 | 1.2 |
| oven curing (24 h/40° C.) | 0.99 | 1.4 |
| microwave 450 Watt; 1 min. | 0.79 | 1.2 |
| microwave 450 Watt; 3 min. | 1.00 | 1.3 |
| microwave 900 Watt; 1 min. | 0.48 | 1.2 |
| ultrasonic 40° C.; 30 min. | 0.61 | 1.7 |
| ultrasonic 40° C.; 45 min. | 0.81 | 1.3 |
| ultrasonic 60° C.; 3 min. | 0.69 | 1.3 |
| ultrasonic 60° C.; 5 min. | 0.53 | 1.3 |
| ultrasonic 80° C.; 1 min. | 0.64 | 1.2 |

### Result:

The LOD values of the uncured material as well as for the cured material are rather low and in the same range. Therefore the curing or stabilizing procedure has apparently no detectable influence on the drying of the film coating rather than on interdiffusion effects in the polymer film structure as discussed before.

Based on the application of microwaves and ultrasonic some coated and cured pellets were stored open and closed in HDPE containers up to 6 month at 25° C./60% relative humidity (r.h.). The drug release profiles were compared to the initial values. Table 5 and 6 show the drug release profiles as well as the investigated LOD's of the stored samples.

TABLE 5
Storage stability; drug release & loss on drying of pellets [%] after 1 min.
microwave curing at 900 Watt

| | 900 Watt/ | 900 Watt/1 min, | 900 Watt/1 min, stored |
|---|---|---|---|
| Time | 1 min, | stored for 1 month | for 1 month 25° C./60% |
| [min] | initial | 25° C./60% r.h. (open) | r.h. (closed) |
| 0 | 0.00 | 0.00 | 0.00 |
| 120 | 1.00 | 0.96 | 1.07 |
| 180 | 7.05 | 6.94 | 5.36 |
| 240 | 47.87 | 41.30 | 37.25 |
| 300 | 83.63 | 77.19 | 76.29 |
| 360 | 94.01 | 90.56 | 91.35 |
| 420 | 97.57 | 95.68 | 96.44 |
| 480 | 98.69 | 97.41 | 98.29 |
| LOD [%] | 0.48 | 0.51 | 0.53 |

### Results:

The drug release profiles as well as the LOD's of the stored and the not stored pellets (initial values) are in the same range. Therefore the application of microwave irradiation with 900 Watt/1 min results in successfully stabilized active ingredient release profiles which are remain their stability even during storage.

TABLE 6
Storage stability; drug release & loss on drying of pellets [%] after 30 min.
cured at 40° C. via ultrasonic

| | Ultrasonic | | Ultrasonic |
|---|---|---|---|
| | 40° C./30 min | Ultrasonic 40° C./30 min | 40° C./30 min stored |
| Time | 1 min, | stored for 1 month | for 1 month 25° C./ |
| [min] | initial | 25° C./60% r.h. (open) | 60% r.h. (closed) |
| 0 | 0.00 | 0.00 | 0.00 |
| 120 | 1.84 | 1.86 | 1.83 |
| 180 | 10.15 | 9.46 | 9.40 |
| 240 | 50.25 | 48.80 | 49.83 |
| 300 | 84.64 | 78.54 | 81.33 |
| 360 | 94.58 | 90.63 | 92.76 |
| 420 | 98.21 | 95.62 | 97.01 |
| 480 | 99.46 | 97.43 | 98.63 |
| LOD | 0.61 | 0.66 | 0.60 |
| [%] | | | |

### Results:

The drug release profiles as well as the LOD's of the stored and the not stored pellets (initial values) are in the same range. Therefore the application of ultrasonic wave irradiation at 40° C. (temperature of the surrounding water) for 30 minutes results in successfully stabilized active ingredient release profiles which are remain their stability even during storage.

### III. Example 2 (According to the Invention)

### Propranolol Pellets Coated with EUDRAGIT® NM 30 D

1,000 g Propranolol HCl pellets (1,000-1,500 μm; 40% drug content) by Lee Pharma Ltd. (Hyderabad, India) were coated with EUDRAGIT® NM 30D by Evonik Industries AG (Darmstadt, Germany), using formulation table 7.

TABLE 7
Coating formulation

| | [g] | [%] on polymer |
|---|---|---|
| EUDRAGIT ® NM 30D | 400.0 | |
| Talc | 120.0 | 100 |
| Hydroxypropyl methylcellulose (HPMC) | 12.0 | 10 |
| Polysorbate 80 (33% aqu.) | 36.4 | 10 |
| Water (Aqua purificata) | 751.6 | |
| | 1,320.0 | |
| Solid content [%] | | 20 |
| Polymer applied [%] | | 12 |

⅓ water (Aqua purificata) was heated up to 45-55° C. Hydroxypropyl methylcellulose (HPMC) by JRS PHARMA GmbH & Co. KG (Rosenberg, Germany) was homogenized with a high shear force (e.g. Ultra Turrax) in the heated Aqua purificata until a clear solution was achieved. When a clear solution was formed, polysorbate 80 (Tween® 80) by Merck KGaA (Darmstadt, Germany) and talc by Merck KGaA (Darmstadt, Germany) were added and dispersed for at least 10 minutes, using the high shear force. Afterwards the excipient suspension was added slowly into the polymer dispersion while stirring with a conventional propeller stirrer. Finalized the suspension was passed through a 240 μm sieve to control that the suspension was free of agglomerates. During the coating process the suspension was gently stirred.

The coating parameters are listed in table 8.

TABLE 8
Equipment set up: Glatt GPCG 1.1 fluid bed coater, top spray mode by
Glatt Process Technology GmbH (Binzen, Germany)

| | | |
|---|---|---|
| Batch size core material | (g) | 1,000.0 |
| Nozzle bore | (mm) | 1.0 |
| Distance bed/spray gun | (cm) | ~10 |
| Internal tube diameter | (mm) | 2.0 |

Process parameter set up:

| | | |
|---|---|---|
| Atomizing air pressure | (bar) | 2.0 |
| Filter cleaning interval | (s) | 60 |
| Filter cleaning time | (s) | 5 |
| Drying air volume | (m³/h) | 70-85 |
| Drying air capacity | (m³/min/kg) | 1.2-1.4 |

Process data:

| | | |
|---|---|---|
| Inlet air temperature | (° C.) | 35-40 |
| Exhaust air temperature | (° C.) | 22-25 |
| Product temperature | (° C.) | 21-23 |
| Spray rate | (g/min/kg) | 11 |
| Spraying time | (min) | ~120 |
| Drying | (min) | 5 |

Table 9 shows the drug release profiles of cured coated pellets; using USP 35-NF30 <724> Drug Release and <711> Dissolution described methods for the determination of the release profile and permissible tolerances. The investigations of the drug release were carried out in an apparatus 2, e.g. Erweka DT 708/1000B by ERWEKA GmbH (Heusenstamm, Germany) in 0.1 N hydrochloric acid, pH 1.2, for the first 120 min, followed by buffer pH 6.8 at 100 rpm, for the rest of the time. The amount of drug released was measured using an UV spectroscope with a wavelength of 289 nm in 914 ml total volume.

TABLE 9
Influence curing conditions on drug release [%]

| Time | | 24 h 40° C. air | 3 min. 450 Watt | 3 min. 900 Watt |
|---|---|---|---|---|
| [min] | uncured | oven | microwave | microwave |
| 0 | 0.00 | 0.00 | 0.00 | 0.00 |
| 120 | 10.10 | 5.62 | 4.44 | 4.71 |
| 180 | 24.67 | 17.86 | 16.17 | 16.54 |
| 240 | 40.02 | 30.56 | 28.98 | 29.00 |
| 300 | 54.14 | 43.13 | 41.69 | 41.46 |
| 420 | 74.34 | 63.65 | 63.43 | 62.26 |
| 540 | 85.05 | 75.40 | 76.45 | 74.84 |
| 660 | 91.37 | 84.19 | 84.29 | 82.32 |

### Results:

Microwave curing with 450 Watt/3 min with 900 Watt/3 min result in a very similar active ingredient release (propranolol) release as the standard method air oven curing 24 h/40° C. Therefore the application of microwave irradiation 450 Watt/3 min or with 900 Watt/3 min results in successfully stabilized active ingredient release profiles.

### IV. Example 3 (According to the Invention)

### Diprophylline Tablets Coated with EUDRAGIT® L 30 D-55

2,000 g Dipropylline tablets (10 mm; round; curvature radius 12 mm; average hardness 85 N; average diameter 335 mg; average height 5 mm; 60% drug content, that means 200 mg/tablet; drug by NBS Biologicals Ltd., Huntingdon, Cambridgeshire UK) prepared by Evonik Industries AG (Darmstadt, Germany), using the tablet formulation table 10 were coated with EUDRAGIT® FS 30D by Evonik Industries AG (Darmstadt, Germany), using the coating formulation of table 11.

TABLE 10
Tablet formulation

| | [%] | [mg/tablet] |
|---|---|---|
| Diprophylline BP | 60.0 | 200.00 |
| Lactose monohydrate (CapsuLac 60) | 12.0 | 40.00 |
| Microcrystalline Cellulose (Avicel ® PH 200) | 12.0 | 40.00 |
| Sodium starch glycolate type A (Explotab ®) | 10.0 | 34.00 |
| Polyvinyl pyrrolidone, PVP (Kollidon ® 25) | 5.0 | 17.00 |
| Magnesium stearate | 1.0 | 4.00 |
| Total | 100.0 | 335.00 |

Diprophylline BP by NBS Biologicals Ltd. (Huntingdon, Cambridgeshire UK), CapsuLac 60 by Molkerei Meggle Wasserburg GmbH & Co. KG (Wasserburg, Germany), Avicel® PH 200 by FMC International (Wallingstown, Ireland), Explotab® by JRS PHARMA GmbH & Co. KG (Rosenberg, Germany) and Kollidon®25 by BASF (Ludwigshafen, Germany) were wet granulated with 400 g water (Aqua purificata), using kitchen machine Kenwood PM900 by Kenwood Limited (Hampshire, UK) followed by hand sieving (2.5 mm mash size) and dried for 20 hours at 40° C. in a circulating air oven by EHRET GmbH & Co. KG (Emmendingen, Germany). Finally magnesium stearate by Merck KGaA (Darmstadt, Germany) was mixed to the granules, using a blender by Servolift GmbH (Offenburg, Germany). Then tablets were compressed, using a rotary tablet press machine E 150 Plus by IMA KILIAN GmbH & Co. KG (Köln, Germany).

TABLE 11
Coating formulation

| | [g] | [%] on polymer |
|---|---|---|
| EUDRAGIT ® L 30D-55 | 301.0 | |
| Triethyl citrate (TEC) | 9.0 | 10 |
| Talc | 45.1 | 50 |
| Water (Aqua purificata) | 367.2 | |
| Total | 722.3 | |
| Solid content [%] | | 20 |
| Polymer applied [mg/cm²] | | 5 |

Triethyl citrate (TEC) and Talc by Merck KGaA (Darmstadt, Germany) were homogenized in water (Aqua purificata) for 15 minutes, using a high shear force (e.g. Ultra Turrax) and added to the polymer dispersion while stirring gently with a conventional propeller stirrer. The final suspension was passed through a 240 μm mm sieve to control that the suspension was free of agglomerates. During the coating process the suspension was gently stirred. The coating parameters are listed in table 12.

TABLE 12
Equipment set up: drum coater O'Hara LabCoat by O'Hara
Technologies Inc. (Toronto, Canada), using 12″ pan size

| | | |
|---|---|---|
| Batch size core material | (g) | 2,000.0 |
| Nozzle bore | (mm) | 1.2 |
| Distance bed/spray gun | (cm) | ~10 |
| Internal tube diameter | (mm) | 2.0 |

Process parameter set up:

| | | |
|---|---|---|
| Pan speed | (rpm) | 18 |
| Atomizing air pressure | (bar) | 1.0 |
| Flat pattern pressure | (bar) | 1.0 |
| Drying air volume | (m³/h) | 150 |

Process data:

| | | |
|---|---|---|
| Inlet air temperature | (° C.) | 44-46 |
| Exhaust air temperature | (° C.) | 33-35 |
| Product temperature | (° C.) | ~30 |
| Spray rate | (g/min/kg) | 5-7 |
| Spraying time | (min) | ~80 |
| Drying | (min) | 3 |

Tables 13-15 shows the drug release profiles as well as the loss on drying (LOD) of cured coated tablets directly after curing and compared to the reference (24 hours at 40° C. in an air oven) using USP 35-NF30 <724> Drug Release and <711> Dissolution described methods for the determination of the release profile and permissible tolerances. The investigations of the drug release were carried out in an apparatus 2, e.g. Erweka DT 708/1000B by ERWEKA GmbH (Heusenstamm, Germany) in 0.1 N hydrochloric acid, pH 1.2, for the first 120 minutes, followed by 2 hours in phosphate buffer pH 6.8 at 100 rpm for the rest of the time. The amount of drug released was measured using an HPLC method (Column: Agilent Zorbax Eclipse XDB C18 column, 150×4.6 mm, 5 μm or equivalent; Mobile Phase: Buffer: Acetonitrile: (65:35); Injection Volume: 10 μL; Flow rate: 1 mL/minute) with a wavelength of 220 nm in 900 ml total volume.

The determination of the LOD was carried out with the Moisture Analyzer HG 63 by Mettler-Toledo GmbH (Gieβen, Germany). 3 g crushed tablets were heated up to 110° C. and dried until mass constancy was achieved.

TABLE 13
Influence curing conditions on drug release [%]; microwave curing compared
to the reference (air oven curing, 24 hours at 40° C.)

| | oven | microwave curing | | | | | | | | | |
|---|---|---|---|---|---|---|---|---|---|---|---|
| | curing | 1 min. | 3 min. | 5 min. | 6 min. | 1 min. | 3 min. | 5 min. | 6 min. | 1 min. | 3 min. |
| Time | 24 h | at 450 | at 450 | at 450 | at 450 | at 630 | at 630 | at 630 | at 630 | at 900 | at 900 |
| (Hr.) | oven | Watt | Watt | Watt | Watt | Watt | Watt | Watt | Watt | Watt | Watt |
| 0.0 | 0.00 | 0.00 | 0.00 | 0.00 | 0.00 | 0.00 | 0.00 | 0.00 | 0.00 | 0.00 | 0.00 |
| 1.0 | 0.00 | 0.00 | 0.38 | 0.00 | 0.00 | 0.00 | 0.00 | 0.00 | 0.00 | 0.00 | 0.00 |
| 2.0 | 0.00 | 0.00 | 0.06 | 0.00 | 0.00 | 0.00 | 0.00 | 0.00 | 0.78 | 0.00 | 0.00 |
| 2.5 | 95.73 | 97.00 | 94.82 | 97.68 | 98.42 | 98.06 | 99.49 | 94.26 | 95.39 | 93.47 | 94.00 |
| 3.0 | 100.41 | 101.97 | 101.60 | 101.78 | 100.98 | 102.45 | 103.67 | 100.47 | 105.58 | 98.40 | 102.51 |
| 3.5 | 100.67 | 102.82 | 102.36 | 102.17 | 101.60 | 102.88 | 103.95 | 100.53 | 105.86 | 98.77 | 102.94 |
| 4.0 | 100.72 | 102.60 | 102.24 | 102.40 | 102.14 | 102.69 | 104.10 | 100.55 | 106.22 | 98.54 | 102.80 |
| LOD | 1.62 | 1.99 | 2.08 | 2.17 | 2.15 | 2.02 | 2.09 | 2.08 | 2.06 | 1.97 | 2.00 |
| [%] | | | | | | | | | | | |

LOD [%] uncured tablets: 1.97

TABLE 14
Influence curing conditions on drug release [%]; ultrasonic curing compared
to the reference (air oven curing, 24 hours at 40° C.)

| | oven | ultrasound curing | | | | | | | | |
|---|---|---|---|---|---|---|---|---|---|---|
| | curing | 5 min. | 15 min. | 30 min. | 60 min. | 120 min. | 1 min. | 3 min. | 5 min. | 120 min. |
| Time | 24 h | at 40° | at 40° | at 40° | at 40° | at 40° | at 80° | at 80° | at 80° | at 80° |
| (Hr.) | oven | C. | C. | C. | C. | C. | C. | C. | C. | C. |
| 0.0 | 0.00 | 0.00 | 0.00 | 0.00 | 0.00 | 0.00 | 0.00 | 0.00 | 0.00 | 0.00 |
| 1.0 | 0.00 | 0.00 | 0.00 | 0.00 | 0.00 | 0.00 | 0.00 | 0.00 | 0.00 | 0.00 |
| 2.0 | 0.00 | 0.00 | 0.00 | 0.00 | 0.00 | 0.00 | 0.00 | 0.00 | 0.00 | 0.00 |
| 2.5 | 95.73 | 96.21 | 93.96 | 99.64 | 91.50 | 97.95 | 98.48 | 98.81 | 94.78 | 95.80 |
| 3.0 | 100.41 | 101.90 | 101.46 | 102.96 | 101.23 | 102.26 | 103.44 | 103.75 | 100.25 | 100.89 |
| 3.5 | 100.67 | 101.93 | 100.68 | 103.02 | 101.47 | 102.25 | 103.35 | 103.17 | 100.28 | 100.99 |
| 4.0 | 100.72 | 102.78 | 101.06 | 103.17 | 101.66 | 102.46 | 103.41 | 103.26 | 100.26 | 101.28 |
| LOD | 1.62 | 2.17 | 2.04 | 2.07 | 2.14 | 2.11 | 2.02 | 2.12 | 1.96 | 2.14 |
| [%] | | | | | | | | | | |

LOD [%] uncured tablets: 1.97

TABLE 15
Influence curing conditions on drug release [%]; IR and UV curing compared
to the reference (air oven curing, 24 hours at 40° C.)

| | | oven curing | | | | | | |
|---|---|---|---|---|---|---|---|---|
| | | IR curing | | | | | | |
| Time | 24 h | 5 min. | 15 min. | 30 min. | UV curing | | | |
| (Hr.) | oven | IR | IR | IR | 5 min. | 15 min. | 30 min. | 120 min. |
| 0.0 | 0.00 | 0.00 | 0.00 | 0.00 | 0.00 | 0.00 | 0.00 | 0.00 |
| 1.0 | 0.00 | 0.00 | 0.00 | 0.00 | 2.10 | 0.00 | 0.00 | 0.00 |
| 2.0 | 0.00 | 0.00 | 0.00 | 0.00 | 2.61 | 0.00 | 0.00 | 0.00 |
| 2.5 | 95.73 | 96.68 | 96.08 | 92.30 | 100.31 | 100.04 | 97.96 | 98.18 |
| 3.0 | 100.41 | 102.90 | 101.88 | 100.30 | 102.46 | 103.10 | 101.95 | 101.43 |
| 3.5 | 100.67 | 102.46 | 102.00 | 100.42 | 102.83 | 103.24 | 101.46 | 101.33 |
| 4.0 | 100.72 | 103.06 | 101.79 | 100.35 | 102.26 | 102.67 | 101.81 | 101.34 |
| LOD | 1.62 | 1.94 | 2.58 | 2.67 | 2.15 | 2.32 | 2.06 | 2.01 |
| [%] | | | | | | | | |

LOD [%] uncured tablets: 1.97

### Results:

Microwave curing with 450 Watt for 1, 3, 5 and 6 minutes, 630 Watt for 1, 3, 5 and 6 minutes and 900 Watt for 1 and 3 minutes or ultrasound curing at 40° C. (temperature of the surrounding water) for 5, 15, 30, 60 and 120 minutes and 80° C. (temperature of the surrounding water) for 1, 3, 5 and 120 minutes or IR curing for 5, 15 and 30 minutes or UV curing for 5, 15, 30 and 120 minutes result in similar active ingredient release (diprophylline) release as the standard method air oven curing 24 h/40° C. Therefore the application of microwave irradiation with 450, 630 and 900 Watt or ultrasonic irradiation at 40 and 60° C. or IR or UV irradiation results in successfully stabilized active ingredient release profiles.

Furthermore, the similar loss on drying (LOD) of uncured and cured coated tablets confirm the curing and disprove a drying process.

The coated and cured tablets were stored closed in HDPE containers up to 3 months at 25° C./60% relative humidity (r.h.), 30° C./65% r.h. and 40° C./75% r.h. The drug release profiles were compared to the initial values.

Results after 3 Month Stored at 40° C./75% r.h.:

Microwave curing with 450 Watt for 1, 3, 5 and 6 minutes, 630 Watt for 1, 3, 5 and 6 minutes and 900 Watt for 1 and 3 minutes or ultrasound curing at 40° C. (temperature of the surrounding water) for 5, 15, 30, 60 and 120 minutes and 80° C. (temperature of the surrounding water) for 1, 3, 5 and 120 minutes or IR curing for 5, 15 and 30 minutes or UV curing for 5, 15, 30 and 120 minutes result in similar active ingredient release (diprophylline) release as the initial values and the reference (24 hours at 40° C.) which are remain their stability even during storage.

### V. Example 4 (According to the Invention)

### Diprophylline Tablets Coated with EUDRAGIT® FS 30 D

2,000 g Dipropylline tablets (10 mm; round; curvature radius 12 mm; average hardness 85 N; average diameter 335 mg; average height 5 mm; 60% drug content, that means 200 mg/tablet; drug by NBS Biologicals Ltd., Huntingdon, Cambridgeshire UK) prepared by Evonik Industries AG (Darmstadt, Germany), using the tablet formulation table 16 were coated with EUDRAGIT® FS 30D by Evonik Industries AG (Darmstadt, Germany), using the coating formulation of table 17.

TABLE 16
Tablet formulation

| | [%] | [mg/tablet] |
|---|---|---|
| Diprophylline BP | 60.0 | 200.00 |
| Lactose monohydrate (CapsuLac 60) | 12.0 | 40.00 |
| Microcrystalline Cellulose (Avicel ® PH 200) | 12.0 | 40.00 |
| Sodium starch glycolate type A (Explotab ®) | 10.0 | 34.00 |
| Polyvinyl pyrrolidone, PVP (Kollidon ® 25) | 5.0 | 17.00 |
| Magnesium stearate | 1.0 | 4.00 |
| Total | 100.00 | 335.00 |

Diprophylline BP by NBS Biologicals Ltd. (Huntingdon, Cambridgeshire UK), CapsuLac 60 by Molkerei Meggle Wasserburg GmbH & Co. KG (Wasserburg, Germany), Avicel® PH 200 by FMC International (Wallingstown, Ireland), Explotab® by JRS PHARMA GmbH & Co. KG (Rosenberg, Germany) and Kollidon®25 by BASF (Ludwigshafen, Germany) were wet granulated with 400 g water (Aqua purificata), using kitchen machine Kenwood PM900 by Kenwood Limited (Hampshire, UK) followed by hand sieving (2.5 mm mash size) and dried for 20 hours at 40° C. in a circulating air oven by EHRET GmbH & Co. KG (Emmendingen, Germany). Finally magnesium stearate by Merck KGaA (Darmstadt, Germany) was mixed to the granules, using a blender by Servolift GmbH (Offenburg, Germany). Then tablets were compressed, using a rotary tablet press machine E 150 Plus by IMA KILIAN GmbH & Co. KG (Köln, Germany).

TABLE 17
Coating formulation

| | [g] | [%] on polymer |
|---|---|---|
| EUDRAGIT ® FS 30D | 361.2 | |
| Triethyl citrate (TEC) | 5.4 | 5 |
| Talc | 54.2 | 50 |
| Water (Aqua purificata) | 419.0 | |
| Total | 839.8 | |
| Solid content [%] | | 20 |
| Polymer applied [mg/cm²] | | 6 |

Triethyl citrate (TEC) and Talc by Merck KGaA (Darmstadt, Germany) were homogenized in water (Aqua purificata) for 15 minutes, using a high shear force (e.g. Ultra Turrax) and added to the polymer dispersion while stirring gently with a conventional propeller stirrer. The final suspension was passed through a 240 μm mm sieve to control that the suspension was free of agglomerates. During the coating process the suspension was gently stirred. The coating parameters are listed in table 18.

TABLE 18
Equipment set up: drum coater by O'Hara Technologies
Inc. (Toronto, Canada), using 12″ pan size

| | | |
|---|---|---|
| Batch size core material | (g) | 2,000.0 |
| Nozzle bore | (mm) | 1.2 |
| Distance bed/spray gun | (cm) | ~10 |
| Internal tube diameter | (mm) | 2.0 |
| Process parameter set up: | | |
| Pan speed | (rpm) | 18 |
| Atomizing air pressure | (bar) | 1.0 |
| Flat pattern pressure | (bar) | 1.0 |
| Drying air volume | (m³/h) | 150 |
| Process data: | | |
| Inlet air temperature | (° C.) | 43-44 |
| Exhaust air temperature | (° C.) | 32-35 |
| Product temperature | (° C.) | 29-33 |
| Spray rate | (g/min/kg) | 5-7 |
| Spraying time | (min) | ~75 |
| Drying | (min) | 3 |

Tables 19 shows the drug release profiles as well as the loss on drying (LOD) of cured coated tablets directly after curing and compared to the reference (24 hours at 40° C. in an air oven) using USP 35-NF30 <724> Drug Release and <711> Dissolution described methods for the determination of the release profile and permissible tolerances. The investigations of the drug release were carried out in an apparatus 2, e.g. Erweka DT 708/1000B by ERWEKA GmbH (Heusenstamm, Germany) in 0.1 N hydrochloric acid, pH 1.2, for the first 120 minutes, followed by 1 hour in phosphate buffer pH 6.8 followed by phosphate buffer pH 7.5 at 100 rpm for the rest of the time. The amount of drug released was measured using an HPLC method (Column: Agilent Zorbax Eclipse XDB C18 column, 150×4.6 mm, 5 μm or equivalent; Mobile Phase: Buffer: Acetonitrile: (65:35); Injection Volume: 10 μL; Flow rate: 1 mL/minute) with a wavelength of 220 nm in 900 ml total volume.

The determination of the LOD was carried out with the Moisture Analyzer HG 63 by Mettler-Toledo GmbH (Gieβen, Germany). 3 g crushed tablets were heated up to 110° C. and dried until mass constancy was achieved.

TABLE 19
Initial values; drug release & loss on drying of tablets [%]

| | | | microwave | | | | | | | | |
|---|---|---|---|---|---|---|---|---|---|---|---|
| | | reference | 450 | 450 | 450 | 900 | 900 | ultrasonic | | | |
| | | oven | W | W | W | W | W | 40° C. | 80° C. | IR | UV |
| | Time | 24 h/ | 1 | 3 | 4 | 1 | 2 | 15 | 3 | 5 | 30 |
| | [min] | 40° C. | min | min | min | min | min | min | min | min | min |
| 0.1N HCl | 0 | 0.0 | 0.0 | 0.0 | 0.0 | 0.0 | 0.0 | 0.0 | 0.0 | 0.0 | 0.0 |
| 0.1N HCl | 60 | 0.0 | 0.0 | 0.0 | 0.0 | 0.0 | 0.0 | 0.0 | 0.0 | 0.0 | 0.0 |
| 0.1N HCl | 120 | 0.0 | 0.0 | 0.0 | 0.0 | 0.0 | 0.0 | 0.0 | 0.0 | 0.0 | 0.0 |
| pH 6.8 | 150 | 0.1 | 0.0 | 0.0 | 0.0 | 0.0 | 0.0 | 0.0 | 0.0 | 0.0 | 0.0 |
| pH 6.8 | 180 | 0.0 | 0.0 | 0.0 | 0.0 | 0.0 | 0.0 | 0.0 | 0.0 | 0.0 | 0.1 |
| pH 7.5 | 210 | 32.5 | 25.2 | 23.2 | 26.2 | 33.8 | 29.8 | 28.7 | 34.9 | 40.9 | 28.3 |
| pH 7.5 | 240 | 101.2 | 99.3 | 97.6 | 96.7 | 96.9 | 97.3 | 98.1 | 100.5 | 100.7 | 98.6 |
| LOD [%] | | 1.96 | 1.87 | 1.99 | 2.05 | 1.95 | 1.99 | 2.15 | 2.11 | 1.93 | 2.10 |

LOD [%] uncured tablets: 1.89

### Results:

The deviation of less than ±10% indicates that all in table 19 listed curing methods are suitable to achieve similar release profiles compared to the reference. Furthermore, the loss on drying (LOD) confirm the curing effect and show that it is definitive a curing and not a drying process.

Some coated and cured tablets were stored in closed HDPE bottles up to 3 months at 25° C./60% r.h., 30° C./60% r.h. as well as 40° C./75% r.h. The drug release was compared to the initial values and to the reference (24 hours at 40° C. air oven curing).

Results after 3 Months Stored at 40° C./75% r.h.:

The deviation of less than 10% indicates that microwave curing with 450 Watt for 1 and 3 minutes or ultrasound curing at 40° C. (temperature of the surrounding water) for 15 minutes and 80° C. (temperature of the surrounding water) for 3 minutes or IR curing for 5 minutes or UV curing for 30 minutes result in similar active ingredient release (diprophylline) release as the initial values and the reference (24 hours at 40° C.) which are remain their stability even during storage.

### VI. Example 5 (According to the Invention)

### Propranolol Pellets Coated with Kollicoat® SR 30 D

1,300 g Propranolol HCl pellets (1,000-1,500 μm; 40% drug content) by Lee Pharma Ltd. (Hyderabad, India) were coated with Kollicoat® SR 30D by BASF AG (Ludwigshafen, Germany), using formulation table 20.

TABLE 20
Coating formulation

| | [g] | [%] on polymer |
|---|---|---|
| Kollicoat ® SR 30D | 433.3 | |
| Talc | 45.5 | 45.5 |
| Triethyl citrate (TEC) | 13.0 | 13.0 |
| Water (Aqua purificata) | 450.7 | |
| Total | 942.5 | |
| Solid content [%] | | 20 |
| Polymer applied [%] | | 10 |

Triethyl citrate (TEC) and Talc by Merck KGaA (Darmstadt, Germany) were homogenized in water (Aqua purificata) for 15 minutes, using a high shear force (e.g. Ultra Turrax) and added to the polymer dispersion while stirring gently with a conventional propeller stirrer. The final suspension was passed through a 240 μm mm sieve to control that the suspension was free of agglomerates. During the coating process the suspension was gently stirred. The coating parameters are listed in table 21.

TABLE 21
Equipment set up: Glatt GPCG 1.1 fluid bed coater, top spray mode by
Glatt Process Technology GmbH (Binzen, Germany)

| | | |
|---|---|---|
| Batch size core material | (g) | 1,300.0 |
| Nozzle bore | (mm) | 1.2 |
| Distance bed/spray gun | (cm) | ~10 |
| Internal tube diameter | (mm) | 2.0 |

Process parameter set up:

| | | |
|---|---|---|
| Atomizing air pressure | (bar) | 2.0 |
| Drying air volume | (m³/h) | 76-84 |

Process data:

| | | |
|---|---|---|
| Inlet air temperature | (° C.) | ~50 |
| Exhaust air temperature | (° C.) | ~30 |
| Product temperature | (° C.) | ~30 |
| Spray rate | (g/min/kg) | 12-14 |
| Spraying time | (min) | 62 |
| Drying | (min) | 5 |

Table 22 and 23 shows the drug release profiles of cured coated pellets; using USP 35-NF30 <724> Drug Release and <711> Dissolution described methods for the determination of the release profile and permissible tolerances. The investigations of the drug release were carried out in an apparatus 2, e.g. Erweka DT 708/1000B by ERWEKA GmbH (Heusenstamm, Germany) in 0.1 N hydrochloric acid, pH 1.2, for the first 120 min, followed by buffer pH 6.8 at 100 rpm, for the rest of the time. The amount of drug released was measured using an HPLC method (Column: Agilent Zorbax Eclipse XDB C18 column, 150×4.6 mm, 5 μm or equivalent; Mobile Phase: Buffer: Acetonitrile: (65:35); Injection Volume: 10 μL; Flow rate: 1 mL/minute) with a wavelength of 220 nm in 900 ml total volume.

The determination of the LOD was carried out with the Moisture Analyzer HG 63 by Mettler-Toledo GmbH (Gieβen, Germany). 3 g crushed tablets were heated up to 110° C. and dried until mass constancy was achieved.

TABLE 22
Initial values; drug release & loss on drying of pellets [%]; microwave curing
compared to the reference (24 hours at 40° C. air oven curing)

| | | Microwave | Microwave | Microwave | Microwave |
|---|---|---|---|---|---|
| | | 450 W 1 min, | 630 W 1 min, | 900 W 1 min, | 900 W 1.5 min, |
| | 24 h/40° C., initial | initial | initial | initial | initial |
| Time (Hr.) | Mean | Mean | Mean | Mean | Mean |
| 0.0 | 0.0 | 0.0 | 0.0 | 0.0 | 0.0 |
| 0.5 | 0.2 | 0.1 | 0.0 | 0.0 | 0.1 |
| 1.0 | 0.1 | 0.1 | 0.0 | 0.1 | 0.1 |
| 1.5 | 0.1 | 0.2 | 0.1 | 0.2 | 0.2 |
| 2.0 | 0.3 | 0.4 | 0.2 | 0.3 | 0.5 |
| 2.5 | 0.8 | 1.0 | 0.7 | 0.8 | 1.1 |
| 3.0 | 1.5 | 2.0 | 1.3 | 1.4 | 2.2 |
| 4.0 | 9.7 | 13.2 | 7.7 | 8.5 | 12.8 |
| 5.0 | 39.7 | 48.6 | 35.5 | 36.1 | 45.6 |
| 6.0 | 72.5 | 80.6 | 67.6 | 65.7 | 75.9 |
| 7.0 | 86.9 | 93.0 | 83.7 | 83.2 | 88.6 |
| 8.0 | 91.9 | 97.6 | 89.6 | 90.4 | 93.2 |
| LOD [%] | 1.77 | 1.71 | 1.55 | 1.73 | 1.45 |

LOD [%] uncured pellets: 1.75

TABLE 22
Initial values; drug release & loss on drying of pellets [%]; ultrasonic curing compared to the reference (24 hours at 40° C. air oven curing)

| | 24 h/ | Ultrasonic | Ultrasonic | Ultrasonic | Ultrasonic | Ultrasonic | Ultrasonic | Ultrasonic | Ultrasonic | Ultrasonic |
|---|---|---|---|---|---|---|---|---|---|---|
| | 40° C., | 40° C. 5 | 40° C. 15 | 40° C. 30 | 40° C. 60 | 40° C. 120 | 60° C. 5 | 60° C. 10 | 80° C. 1 | 80° C. 2 |
| Time | initial | min, initial | min, initial | min, initial | min, initial | min, initial | min, initial | min, initial | min, initial | min, initial |
| (Hr.) | Mean | Mean | Mean | Mean | Mean | Mean | Mean | Mean | Mean | Mean |
| 0.0 | 0.0 | 0.0 | 0.0 | 0.0 | 0.0 | 0.0 | 0.0 | 0.0 | 0.0 | 0.0 |
| 0.5 | 0.2 | 0.3 | 0.3 | 0.5 | 0.3 | 0.1 | 0.3 | 0.1 | 0.0 | 0.0 |
| 1.0 | 0.1 | 0.2 | 0.1 | 0.2 | 0.1 | 0.1 | 0.6 | 0.2 | 0.0 | 0.0 |
| 1.5 | 0.1 | 0.3 | 0.2 | 0.2 | 0.1 | 0.2 | 0.8 | 0.2 | 0.1 | 0.1 |
| 2.0 | 0.3 | 0.5 | 0.4 | 0.5 | 0.3 | 0.4 | 1.2 | 0.4 | 0.2 | 0.3 |
| 2.5 | 0.8 | 1.6 | 1.5 | 1.1 | 0.8 | 0.9 | 1.8 | 0.9 | 1.2 | 1.2 |
| 3.0 | 1.5 | 1.8 | 1.9 | 1.7 | 2.0 | 1.8 | 3.2 | 1.7 | 1.8 | 1.6 |
| 4.0 | 9.7 | 9.8 | 12.1 | 10.2 | 11.6 | 10.2 | 15.1 | 13.2 | 9.8 | 9.7 |
| 5.0 | 39.7 | 40.3 | 45.0 | 45.6 | 43.2 | 42.1 | 48.4 | 44.1 | 38.3 | 40.3 |
| 6.0 | 72.5 | 71.6 | 76.6 | 77.4 | 73.6 | 73.1 | 74.3 | 74.3 | 71.8 | 72.0 |
| 7.0 | 86.9 | 85.7 | 87.7 | 90.7 | 86.6 | 83.6 | 88.0 | 88.7 | 87.8 | 89.1 |
| 8.0 | 91.9 | 89.7 | 92.1 | 95.2 | 91.1 | 91.7 | 91.7 | 92.2 | 92.7 | 94.0 |
| LOD | 1.77 | 1.96 | 2.10 | 1.93 | 1.96 | 1.78 | 2.05 | 1.94 | 2.01 | 1.88 |
| [%] | | | | | | | | | | |

LOD [%] uncured pellets: 1.75

### Results:

Microwave curing with 450, 630 for 1 minute and 900 Watt for up to 1.5 minutes or ultrasonic curing at 40 (for 5, 15, 30, 60 and 120 minutes), 60 (for 5 and 10 minutes) and 80° C. (water temperature) for 1 and 2 minutes result in a very similar active ingredient release (propranolol) release as the standard method air oven curing 24 h/40° C. Therefore the application of microwave irradiation or ultrasonic irradiation results in successfully stabilized active ingredient release profiles. Furthermore, the loss on drying (LOD) confirm the curing effect and show that it is definitive a curing and not a drying process.

### VII. Comparative Example 1

### Diprophylline Pellets Coated with EUDRAGIT® RS 30 D

Negative results show the samples from example 1 cured with 450 Watt for 5 minutes and at 900 Watt for 3 minutes. During the curing process the pellets sticked together and changed her colour due to the longer curing times.

Furthermore, the samples cured via ultrasonic at 40° C. (water temperature) for 15 minutes, at 60° C. (water temperature) for 10 minutes and at 80° C. (water temperature) for 3 minutes showed instability after one month at 25° C./60% r.h. apparently due to too short or too long curing times.

### VIII. Comparative Example 2

### Diprophylline Tablets Coated with EUDRAGIT® FS 30D

Negative results show the samples from example 4 cured with 450 Watt for 5 minutes and at 900 Watt for 3 minutes. During the curing process the tablets sticked together and the film was swelled due to the longer curing times.

Furthermore, the curing via ultrasonic at 40° C. (water temperature) for 5 minutes and at 80° C. (water temperature) for 1 minutes as well as by UV irradiation for 15 minutes show instabilities after 3 months at 40° C./75% r.h. what indicate, that the curing time was too short to achieve a curing effect.

1: A process for stabilizing the drug release profile of a polymer film coated pharmaceutical or nutraceutical composition, wherein the pharmaceutical composition comprises a core comprising at least one active pharmaceutical ingredient and a polymer film coating onto the core,
the process comprising:
applying the polymer film onto the core by a spray coating process; and
subsequently drying by applying microwave-irradiation, infrared-irradiation, UV-irradiation or ultrasonic wave irradiation to the polymer film-coated pharmaceutical composition.

2: The process according to claim 1, wherein a residual water content of the polymer film coated pharmaceutical composition is less than 10%.

3: The process according to claim 1, wherein microwave-irradiation with frequencies within the range of 300 MHz to less than 300 GHz is applied.

4: The process according to claim 3, wherein microwave-irradiation with frequencies in the range of 2.4 to 2.5 GHz is applied.

5: The process according to claim 1, wherein
infrared-irradiation with frequencies within the range of 300 GHz to 400 THz is applied.

6: The process according to claim 1, wherein ultra sonic wave irradiation with frequencies within the range of 16 kHz to 1 GHz is applied.

7: The process according to claim 1, wherein UV irradiation with frequencies within the range of 789 THz to 300 PHz is applied.

8: The process as claimed in claim 1, wherein the coating of the pharmaceutical form comprises vinyl copolymers, polyvinyl acetate or derivatives of polyvinyl acetate.

9: The process as claimed in claim 8, wherein the coating of the pharmaceutical form comprises (meth)acrylate (co)polymers which are polymerized to more than 95% by weight to 100% from monomers comprising neutral radicals.

10: The process as claimed in claim 9, wherein the (meth)acrylate copolymers are polymerized from 20 to 40% by weight of ethyl acrylate and 60 to 80% by weight of methyl methacrylate.

11: The process as claimed in claim 8, wherein the coating of the pharmaceutical form comprises cationic (meth)acrylate copolymers.

12: The process as claimed in claim 11, wherein the coating of the pharmaceutical form comprises (meth)acrylate copolymers which comprise quaternary ammonium groups.

13: The process as claimed in claim 12, wherein the coatings of the pharmaceutical forms comprises (meth)acrylate copolymers which are synthesized from free radical-polymerized units of 50-70% by weight of methyl methacrylate, 20-40% by weight of ethyl acrylate and 2-12% by weight of 2-trimethylammonium ethyl methacrylate chloride.

14: The process as claimed in claim 11, wherein the coatings of the pharmaceutical form comprises (meth)acrylate copolymers which comprise tertiary amino groups.

15: The process as claimed in claim 14, wherein the coatings of the pharmaceutical forms comprises (meth)acrylate copolymers which are synthesized from 20-30% by weight of methyl methacrylate, 20-30% by weight of butyl methacrylate and 60-40% by weight of dimethylaminoethyl methacrylate.

16: The process as claimed in claim 8, wherein the coating of the pharmaceutical form comprises anionic (meth)acrylate (co)polymers.

17: The process as claimed in claim 16, wherein the coating of the pharmaceutical form comprises anionic (meth)acrylate (co)polymers which consist to 25 to 95% by weight of free radical-polymerized C1- to C4-alkyl esters of acrylic or methacrylic acid and to 5 to 75% by weight of (meth)acrylate monomers comprising an anionic group in the alkyl radical.

18: The process as claimed in claim 1, wherein the process for stabilizing the drug release profiles is carried out in a fluidized bed coating apparatus or in a drum coating apparatus, which are additionally equipped with microwave-irradiation devices, infrared-irradiation device, UV-irradiation devices or ultrasonic wave irradiation devices, where the pellets are coated first, dried and then stabilized in the same apparatus.

19: The process as claimed in claim 1, wherein the microwave-irradiation, the infrared-irradiation, the UV-irradiation or the ultrasonic wave irradiation is applied to the polymer film coated pharmaceutical composition for not more than 60 minutes.
